(19) United States
(12) Patent Application Publication
(10) Pub. No.: US 2009/0137688 A1
(43) Pub. Date: May 28, 2009

(54) PHARMACEUTICAL PRODUCT

(76) Inventor: Hongli Yang, Worcestershire (GB)

Correspondence Address: NIXON & VANDERHYE, PC, 901 NORTH GLEBE ROAD, 11TH FLOOR, ARLINGTON, VA, 22203, US

(21) Appl. No.: 12/158,720

(22) PCT Filed: Dec. 6, 2006
(86) PCT No.: PCT/GB2006/004563

§ 371 Date: Oct. 6, 2008

(30) Foreign Application Priority Data
Dec. 23, 2005 (GB) 0526332.2

(51) Int. Cl. A61K 47/04

(52) U.S. Cl. 514/770

(57) ABSTRACT

The present invention relates to a pharmaceutical product comprising porous silicon, a beneficial substance, and an excipient, the beneficial substance being located in at least some of the pores of the porous silicon, and the excipient having a structure and composition such that it has a melting point between 25 C and 45 C. The invention allows improved control over the release of a beneficial substance from porous silicon.

The present invention relates to a pharmaceutical product comprising silicon. More specifically the present invention relates to a pharmaceutical product comprising silicon, a drug, and an excipient. Yet more specifically the present invention relates to a pharmaceutical product comprising porous silicon, a drug, and an exciptient.

Porous silicon is a form of silicon that typically has porosities between 2% and 99%. The pores have irregular cross-sections, and non-uniform spatial distribution. Porous silicon is most commonly formed by anodisation, or stain etching, of non-porous forms of the element. Anodisation normally results in the formation of a hydrogen terminated, hydrophobic, surface. Upon air ageing, or heating in an oxygen containing atmosphere, Si—O bonds may be formed at the surface of the porous silicon, which tend to confer hydrophilicity.

Forms of porous silicon have been shown to erode in a number of biological environments. For example it has been shown to erode in simulated human plasma (WO 9706101), and it has been shown to erode in simulated intestinal fluid (WO 0128529). It also has a number of other advantageous biological properties, for example it may be bioactive, and biocompatible.

The use of porous silicon for drug delivery has been described in several patent applications. For example WO 9953898 describes the use of porous silicon implants to deliver a beneficial substance; WO 0128529 describes the use of porous silicon to deliver drugs via the gastrointestinal tract; WO 02067998 describes the use of porous silicon to deliver anti-cancer agents to a tumour; and WO 05042023 describes methods for introducing drugs into the pores of the porous silicon. WO 0128529 also describes the use of an excipient coating, which may dissolve in the mouth of a patient before drug delivery occurs.

There are two main mechanisms, by which a medical device comprising porous silicon, may deliver a beneficial substance: (i) the beneficial substance may diffuse through the pores until it reaches the exterior of the device, and (ii) the porous silicon may erode to cause release.

For many drug delivery applications, the rate at which the drug is delivered can play a very significant role in the effectiveness of the treatment. If the drug is to be delivered using porous silicon, then the more mechanisms (i) and (ii) can be controlled, the more useful the delivery means will be.

It is an objective of the present invention to provide a method of improving control of the delivery of a beneficial substance to an animal or human subject using silicon. It is a further objective of the present invention to improve loading of hydrophilic beneficial substances into hydrophobic porous silicon.

According to a first aspect, the invention provides a pharmaceutical product comprising silicon, a beneficial substance, and an excipient.

The silicon may be selected from one or more of: bulk crystalline silicon, polycrystalline silicon, amorphous silicon, porous silicon, bioactive silicon, biocompatible silicon, and resorbable silicon.

The porous silicon may comprise one or more of microporous silicon, macroporous silicon, and mesoporous silicon. The silicon may comprise porous silicon having a porosity between 2% and 99%. The silicon may comprise porous silicon having a porosity between 20% and 60%. The silicon may comprise porous silicon having a porosity between 40% and 85%.

For the absence of doubt, bioactive silicon is silicon that is capable of forming a bond with living tissue when implanted into a living animal or human. Resorbable silicon is silicon that capable of erosion when introduced into or onto a physiological organ, tissue, or fluid of a living human or animal. A “beneficial substance” is something beneficial overall to a human or animal to which it has been administered: it could be a toxin toxic to undesirable cells/to interfere with an undesirable physiological process. For example, anti-cancer substances would be considered “beneficial”, even though their aim is to kill cancer cells. The term pharmaceutical product includes pharmaceutical products that are devices. Microporous silicon is porous silicon having a mean pore size less than 20 A, mesoporous silicon is porous silicon having a mean pore size between 20 A and 500 A, and macroporous silicon is porous silicon having a mean pore size greater than 500 A.

The silicon may comprise silicon having a multiplicity of surface Si—H bonds. The silicon may comprise silicon having a multiplicity of surface Si—O bonds. The silicon may comprise silicon that is substantially completely hydrogen terminated. The silicon may comprise silicon that is substantially completely oxygen terminated. The silicon may have a hydrophobic surface. The silicon may have a hydrophilic surface.

The excipient may have a structure and composition such that it has a melting point between 20 C and 45 C. The excipient may have a structure and composition such that it has a melting point between 30 C and 45 C. The excipient may have a structure and composition such that it has a melting point between 35 C and 40 C. The excipient may have a structure and composition such that it has a melting point between 36 C and 38 C.

The excipient may have a structure and composition such that the excipient is solid and malleable within at least part of the temperature range −50 C and 45 C. The excipient may have a structure and composition such that the excipient is solid and malleable within at least part of the temperature range −10 C and 45 C. The excipient may have a structure and composition such that the excipient is solid and malleable within at least part of the temperature range 0 C and 45 C.

The excipient may have a structure and composition such that it is hydrophobic. The excipient may have a structure and composition such that it is hydrophilic.

The excipient may comprise one or more of: a lauric cocoa butter substitute; a palm kernel oil derivative; a lauric fat that has been hydrogenated to provide a trans acid content of at least 25%; and one or more 1,3-disaturated-2-unsaturated triglycerides or their components.

The excipient may comprise at least 80% 1,3-disaturated-2-oleoyl glycerols which are up to 10% 1,3-dipalmitoyl-2-oleoyl glycerol, 25-45% 1-palmitoyl-2-oleoyl-3-stearoyl glycerol, and 45-70% 1,3-distearoyl-2-oleoyl glycerol.

The excipient may comprise an interesterified mixture of 75-90% lauric acid or oil (including palm kernel oil) and 10-25% non-lauric oil.

The excipient may comprise Palm Kernel Stearin, Hydrogenated Palm Kernel Stearin, and Hardened Palm Oil.

The excipient may comprise Palm Kernel Stearin, Hydrogenated Palm Kernel Stearin, Hardened Palm Oil, Palm Kernel Oil, and Hydrogenated Palm Kernel Oil. The excipient may comprise glycerinated gelatine.

The excipient may comprise PEG.

The excipient may comprise PEG having a molecular weight between 200 and 35000.

The excipient may comprise one or more of: Cocoa butter, hydrogenated Coco-Glycerides-Witepsol Series. The Witepsol Series being produced by the company Witepsol Inc.

The excipient may comprise one or more of: Witepsol H15, Witepsol H32, Witepsol H37, Witepsol H185, Witepsol E 85

The excipient may comprise one or more of: water soluble materials such as glycerinated gelatine, polyethylene glycols; Fatty materials such as triglycerides of palmitic acid, stearic acid, and oleic acid; glycerides of saturated C10 to C18 fatty acids, hydrogenated glycerides.

The excipient may comprise Glycerin and/or gelatine.

The excipient may comprise one or more of: polyethylene glycol 400, polyethylene glycol 8000, polyethylene glycol 1000, polyethylene glycol 3350.

The beneficial substance may have a structure and composition such that it is hydrophobic. The beneficial substance may have a structure and composition such that it is hydrophilic.

At least part of the beneficial substance may distributed throughout the excipient. Substantially all the beneficial substance may be distributed through at least some of the exciptient. Substantially all the beneficial substance may be substantially uniformly distributed through at least part of the excipient. Between 10% and 50% of the beneficial substance may be distributed in one half of the excipient's mass, and the remainder may be distributed in the other half of the excipient's mass. Between 40% and 90% of the beneficial substance may be distributed in one half of the excipient's mass, and the remainder may be distributed in the other half of the excipient's mass.

At least part of the excipient may form a barrier between at least part of the beneficial substance and the exterior of the pharmaceutical product. At least part of the excipient may form a barrier between substantially all the beneficial substance and the exterior of the pharmaceutical product.

The pharmaceutical product may comprise one or more of: an implant, a tablet, a pellet, a powder, a capsule, a suppository, a particle, and an injectable formulation.

The pharmaceutical product may have a largest dimension between 1 micron and 10 microns. The pharmaceutical product may have a largest dimension between 1 micron and 500 microns. The pharmaceutical product may have a largest dimension between 500 microns and 2 cm. The pharmaceutical product may have a largest dimension between 500 microns micron and 5 mm. The pharmaceutical product may have a largest dimension between 1 mm and 5 mm. The pharmaceutical product may have a largest dimension between 1 micron and 2 cm.

The pharmaceutical product may comprise porous silicon, the excipient and the porous silicon being arranged such that at least part of the excipient is located in the pores of the porous silicon. The pharmaceutical product may comprise porous silicon, the excipient and the porous silicon being arranged such that substantially all of the excipient is located in the pores of the porous silicon. The pharmaceutical product may comprise porous silicon, the excipient and the porous silicon being arranged such that at least part of the excipient substantially encloses the porous silicon. The pharmaceutical product may comprise porous silicon, the excipient and the porous silicon being arranged such that at least 50%, by mass, of the excipient is located in the pores of the porous silicon. The pharmaceutical product may comprise porous silicon, the excipient and the porous silicon being arranged such that at least 80%, by mass, of the excipient is located in the pores of the porous silicon.

The pharmaceutical product may comprise a sample of porous silicon, and the excipient may form a coating that substantially surrounds the sample of the porous silicon. The pharmaceutical product may comprise a sample of porous silicon, and the excipient may form a coating that is in contact with at least part of the sample of porous silicon. The depth of the excipient coating may be between 0.01 mm and 2 cm. The depth of the excipient coating may be between 0.1 mm and 2 mm. The depth of the excipient coating may be between 0.5 mm and 1.5 mm.

The pharmaceutical product may comprise porous silicon, at least part of the beneficial substance being located in at least some of the pores of the porous silicon. The pharmaceutical product may comprise porous silicon, at least part of the beneficial substance being located in at least some of the pores of the porous silicon, and the excipient may form a coating that obscures at least some of the pores in which at least some of the beneficial substance is located. The pharmaceutical product may comprise porous silicon, at least part of the beneficial substance being located in at least some of the pores of the porous silicon, and the excipient may form a coating that forms a barrier across at least some of the pores in which at least some of the beneficial substance is located.

The pharmaceutical product may comprise porous silicon, at least part of the beneficial substance and at least part of the excipient being located in at least some of the pores of the porous silicon. The pharmaceutical product may comprise porous silicon, at least part of the beneficial substance and at least part of the excipient being located in at least some of the pores of the porous silicon, part of the beneficial substance being substantially uniformly distributed throughout the pore volume of at least one of the pores. The pharmaceutical product may comprise a sample of porous silicon, at least part of the beneficial substance being located in at least some of the pores of the porous silicon and at least part of the excipient being located in at least some of the pores of the porous silicon, the beneficial substance and the excpitient being arranged such that at least part of the excipient forms a barrier between at least part of the beneficial substance and the exterior of the sample of porous silicon.

The silicon may comprise a silicon particulate product comprising a multiplicity of silicon particles. The silicon may comprise a silicon particulate product comprising between 10 and 10²⁶ silicon particles. The silicon may comprise a silicon particulate product comprising a multiplicity of porous silicon particles. The silicon may comprise a silicon particulate product comprising between 10 and 10³ silicon porous silicon particles. The silicon may comprise a silicon particulate product comprising between 10 and 10⁶ silicon porous silicon particles. The silicon may comprise a silicon particulate product comprising between 10 and 10¹⁰ silicon porous silicon particles. The silicon may comprise a silicon particulate product comprising between 10 and 10¹⁷ silicon porous silicon particles.

Each of the silicon particles from which the particulate product is formed may be coated with part of the excipient. Some of the silicon particles from which the particulate product is formed may be coated with part of the excipient. At least part of the surface of each of the silicon particles from which the particulate product is formed may be coated with part of the excipient.

The silicon particulate product may comprise a multiplicity of excipient coated particles, each of the excipient coated particles having a layer of excipient in contact with at least part of its surface.

The silicon particulate product may comprise a multiplicity of excipient bonded particles, each excipient bonded particle comprising two or more silicon particles that are at least partly bonded together by contact with part of the excipient. At least part of the excipient may form a coating that substantially encloses at least some of the excipient bonded particles.

The silicon particulate product may be distributed through at least part of the excipient. The silicon particulate product may be substantially uniformly distributed through at least part of the excipient.

The silicon particulate product may be distributed through at least part of the excipient, and comprise porous silicon, at least part of the beneficial substance being located in at least some of the pores of the porous silicon.

The silicon particulate product may be distributed through at least part of the excipient, and at least part of the beneficial substance may be located in at least some of the excipient.

The silicon may comprise a bonded silicon particulate product, the bonded product comprising a multiplicity of bonded silicon particles, each bonded silicon particle being bonded to at least one of the other bonded silicon particles.

The silicon may comprise a bonded silicon particulate product, the bonded product comprising a multiplicity of bonded porous silicon particles, each bonded porous silicon particle being bonded to at least one of the other bonded porous silicon particles.

At least part of the bonded silicon particulate product may be in contact with at least part of the excipient. At least part of the excipient may substantially surround the bonded particulate product. At least part of the excipient may form a coating, at least part of which is in contact with at least part of the bonded silicon product.

The bonded silicon particulate product may comprise bonded porous silicon particles, and at least some of the excipient may be located in at least some of the pores of the bonded porous silicon particles. Pores may be formed from the spaces between the bonded silicon particles, and at least part of the excipient may be located in at least some of the pores formed from the spaces between the bonded silicon particles.

The bonded silicon particulate product may comprise a number of bonded porous silicon particles, at least some of the beneficial substance being located in the pores of at least some of the porous silicon particles. The bonded silicon particulate product may comprise a number of bonded porous silicon particles, at least some of the beneficial substance being located in the pores of at least some of the bonded porous silicon particles, and at least some of the excipient being located in at least some of the pores of the bonded porous silicon particles.

The bonded silicon particulate product may comprise a number of bonded silicon particles, at least some of the beneficial substance being located in the pores formed by the spaces between the bonded silicon particles. The bonded silicon particulate product may comprise a number of bonded silicon particles, at least some of the beneficial substance being located in the pores formed by the spaces between the bonded silicon particles, and at least some of the excipient being located in at least some of the pores formed by the spaces between the bonded silicon particles.

The bonded silicon particulate product may comprise a silicon unitary body comprising a multiplicity of integral silicon particles, each of the integral silicon particles being integral with each of the other integral silicon particles. At least part of the excipient may form a coating that substantially encloses the silicon unitary body.

If an excipient acts as a barrier between the beneficial substance and the surroundings of the pharmaceutical product, then this can help to control the rate of release of the beneficial substance. The beneficial substance may be released as a result of diffusion into, and/or through the expient. If the excipient has a melting temperature that is less than or equal to the body temperature of the human or animal into which it has been introduced, then introduction will result in melting of the excipient. Such melting may result in removal of the barrier that prevented the release of the beneficial substance. If the beneficial substance has diffused into the excipient then melting may also result in dispersion of the beneficial substance away from the sample of silicon with which it was associated. If the excipient is hydrophobic the beneficial substance is hydrophobic, then the use of the hydrophobic excipient may encourage transfer of the beneficial substance into the animal or human, as a result of absorption of the beneficial substance into the excipient and consequent dispersion as the excipient is separated from the silicon. Similarly if a beneficial substance is hydrophilic then the use of a hydrophobic excipient, which also acts as a barrier to the beneficial substance, may reduce the rate of the transfer of the beneficial substance to the surroundings of the pharmaceutical product. If the porous silicon has a hydrophobic surface, then combination of a hydrophilic substance with a hydrophobic excipient, followed by introduction of the mixture into the pores of the porous silicon, may facilitate loading of the beneficial substance. If the porous silicon has a hydrophilic surface, then combination of a hydrophobic substance with a hydrophilic excipient, followed by introduction of the mixture into the pores of the porous silicon, may facilitate loading of the beneficial substance.

According to a further aspect the invention provides a method of delivering a beneficial substance to an animal or human comprising the steps:
(a) introducing a pharmaceutical product comprising a beneficial substance and an excipient, as defined in any of the above mentioned aspects, to the animal or human; and
(b) allowing the excipient to melt thereby delivering the beneficial substance.

The method may comprise the further step (c) of allowing at least part of the beneficial substance to pass into the excipient. The step (c) may occur prior to step (a). The step (b) may comprise the step (c).

The method may comprise the step (d) of allowing the beneficial substance to pass through at least part of the excipient to the exterior of the pharmaceutical product. The step (b) may comprise the step (d). The step (d) may occur prior to step (b). The step (d) may occur during step (b). The step (d) may after step (b).

The method may comprise the further step (e) of separating at least part of the excipient from the sample of porous silicon. The step (e) may comprise the step (b). The step (e) may occur after and/or during step (b).

The method may comprise the further step (f) of allowing the excipient to move from the mouth of one or more pores in which at least part of the beneficial substance is located. The step (f) may comprise the step (b). The step (f) may occur after and/or during step (b).

The method may comprise the step (g) of allowing the excipient to move from one or more pores in which the beneficial substance is located. The step (g) may comprise the step (b). The step (g) may occur after and/or during step (b).

For the absence of doubt, the step (b) of allowing the excipient to melt may comprise the step of allowing the excipient to melt when it has been combined with a beneficial substance and/or combined with silicon.

According to a further aspect the invention provides a method of loading a beneficial substance into at least some of the pores of porous silicon, the method comprising the steps: (a) combining the beneficial substance with an excipient to produce an excipient mixture, (b) melting the excipient mixture, (c) allowing the molten excipient mixture to pass into the pores of the porous silicon.

The porous silicon may have a hydrophobic surface and the excipient may have a structure and composition such that it is hydrophobic.

The porous silicon may have a hydrophilic surface and the excipient may have a structure and composition such that it is hydrophilic.

The excipient may have a structure and composition such that it has a melting point between 25 C and 45 C. The excipient may have a structure and composition such that it has a melting point between 30 C and 45 C. The excipient may have a structure and composition such that it has a melting point between 35 C and 40 C. The excipient may have a structure and composition such that it has a melting point between 36 C and 38 C.

The porous silicon may comprise silicon having a multiplicity of surface Si—H bonds. The porous silicon may comprise silicon having a multiplicity of surface Si—H bonds, at least some the hydrogen terminated surface being located in at least some of the pores of the porous silicon. The porous silicon may comprise silicon that is substantially completely hydrogen terminated. Substantially the whole surface of the porous silicon may be hydrophobic. At least part of the porous silicon surface may be oxygen terminated.

The excipient may have a structure and composition such that it is hydrophobic. The excipient may have a structure and composition such that it is hydrophilic.

The excipient may comprise one or more of: a lauric cocoa butter substitute; a palm kernel oil derivative; a lauric fat that has been hydrogenated to provide a trans acid content of at least 25%; and one or more 1,3-disaturated-2-unsaturated triglycerides. The excipient may comprise at least 80% 1,3-disaturated-2-oleoyl glycerols which are up to 10% 1,3-dipalmitoyl-2-oleoyl glycerol, 25-45% 1-palmitoyl-2-oleoyl-3-stearoyl glycerol, and 45-70% 1,3-distearoyl-2-oleoyl glycerol.

The excipient may comprise an interesterified mixture of 75-90% lauric acid or oil (including palm kernel oil) and 10-25% non-lauric oil.

The excipient may comprise Palm Kernel Stearin, Hydrogenated Palm Kernel Stearin, and Hardened Palm Oil.

The excipient may comprise Palm Kernel Stearin, Hydrogenated Palm Kernel Stearin, Hardened Palm Oil, Palm Kernel Oil, and Hydrogenated Palm Kernel Oil.

The excipient may comprise glycerinated gelatine.

The excipient may comprise PEG.

The excipient may comprise PEG having a molecular weight between 200 and 35000.

The excipient may comprise one or more of: Cocoa butter, hydrogenated Coco-Glycerides-Witepsol Series. The Witepsol Series being produced by the company Witepsol Inc.

The excipient may comprise one or more of: Witepsol H15, Witepsol H32, Witepsol H37, Witepsol H185, Witepsol E 85

The excipient may comprise one or more of: water soluble materials such as glycerinated gelatine, polyethylene glycols; Fatty materials such as triglycerides of palmitic acid, stearic acid, and oleic acid; glycerides of saturated C10 to C18 fatty acids, hydrogenated glycerides.

The excipient may comprise one or more of a Glycerin, a gelatine, and Glycerinated gelatine.

The excipient may comprise one or more of: polyethylene glycol 400, polyethylene glycol 8000, polyethylene glycol 1000, polyethylene glycol 3350.

The invention will now be described by way of example only, with reference to the following diagrams:

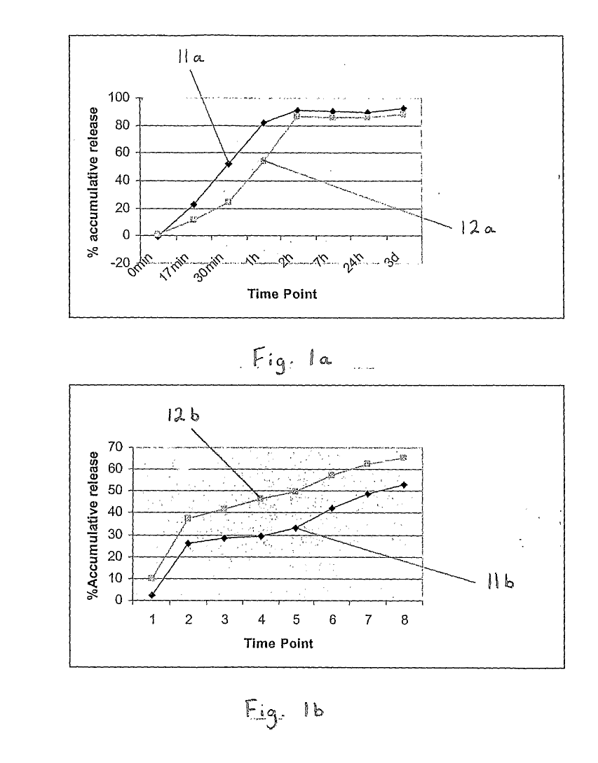

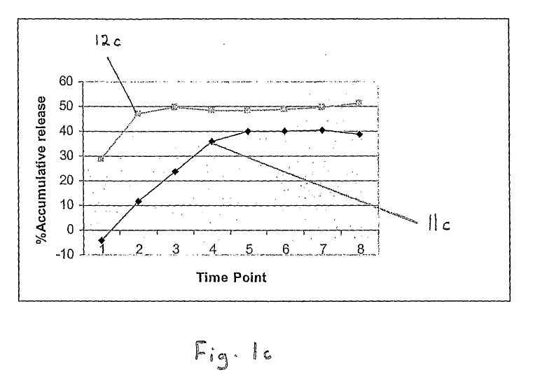

FIG. 1a contains a graph showing the variation of accumulative release with time for porous silicon that has been loaded with chlorambucil and coated with cocoa butter;

FIG. 1b contains a graph showing the variation of accumulative release with time for porous silicon that has been loaded with amitriptyline hydrochloride and coated with cocoa butter;

FIG. 1c contains a graph showing the variation of accumulative release with time for porous silicon that has been loaded with neutral red and coated with cocoa butter;

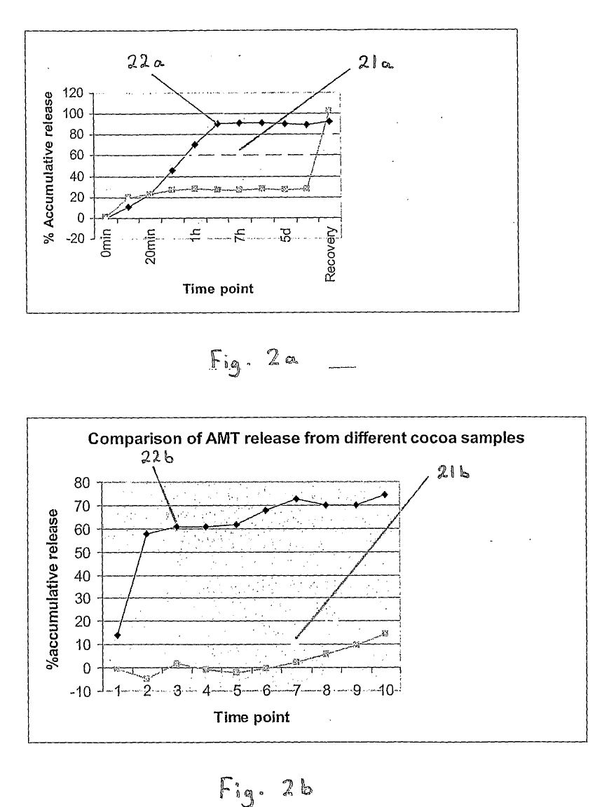

FIG. 2a contains a graph showing the variation of accumulative release with time for porous silicon that has been loaded with chlorambucil and with cocoa butter;

FIG. 2b contains a graph showing the variation of accumulative release with time for porous silicon that has been loaded with amitriptyline hydrochloride and with cocoa butter;

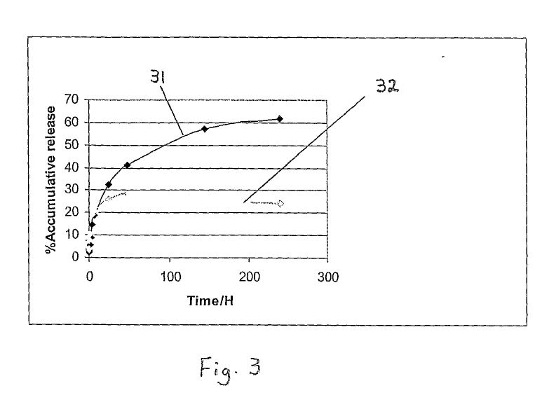

FIG. 3 contains a graph showing the variation of accumulative release with time for a pellet formed by the compression of porous silicon combined with a beneficial substance and cocoa butter.

A number of experiments were performed to show the effect of an excipient upon the release of a beneficial substance from the pores of porous silicon. Three substances were tested: chlorambucil, amitriptyline hydrochloride, neutral red. The excipient employed was cocoa butter. Three methods of combining the porous silicon, excipient, and beneficial substance, were carried out.

Method A comprises the steps of: (a) dissolving the beneficial substance in ethanol; (b) introducing the ethanolic solution of the beneficial substance to the surface of the porous silicon wafer and allowing it to pass into the pores; (c) applying heat to the porous silicon wafer by means of a hot plate to drive off the ethanol, (d) removing the excess beneficial substance, that has not entered the pores, by washing with ethanol; (e) dropping melted cocoa butter oil onto the porous side of the wafer, which has been loaded with a beneficial substance; and (f) allowing the wafer to cool at an ambient temperature of 20 C for approximately one hour, the cocoa butter forming a layer, approximately 1 mm in depth, that coats the surface of the porous silicon.

Method B comprises the steps: (a) dissolving the beneficial substance and excipient in ethanol; (b) introducing the ethanolic solution of the beneficial substance and excipient to the surface of the porous silicon wafer and allowing it to pass into the pores; (c) applying heat to the porous silicon wafer by means of a hot plate thereby driving off the ethanol, and (d) removing the excess beneficial substance and excipient, that have not entered the pores, by washing with ethanol. The porosified wafer, utilized for methods A and B, comprised mesoporous silicon having a porosity of 74%.

Method C comprises the steps: (a) fabricating particles of porous silicon by milling one or more porous silicon membranes; (b) combining the silicon particles with an ethanolic solution of the beneficial substance in a flask; (c) removing the ethanol by rotary evaporation; (d) mixing the beneficial substance loaded particles with excipient, (e) introducing the mixture to a cylindrical die, and (f) applying a uniaxial pressure of 0.5 ton to form a pellet. The porous silicon membrane, utilized for method C, comprises mesoporous silicon having a porosity of: 77%.

Samples of porous silicon that have been combined with beneficial substance and excipient by methods A, B, and C were then immersed in PBS buffer at 37 C, and the accumulative release was measured by means of a UV spectroscopic technique. FIG. 1 shows results for samples prepared by method A, FIG. 2 shows results for samples prepared by method B, and FIG. 3 shows results for a sample prepared by method C.

Graph 11a, of FIG. 1a, shows release of chlorambucil when the porous silicon sample has been coated, and graph 12a shows release of chlorambucil when the wafer is uncoated. The rate of release increases when the wafer has been coated with cocoa butter, relative to the uncoated state. This effect may result from attraction, at a molecular level, between the hydrophobic chlorambucil and the hydrophobic cocoa butter. The cocoa butter melts at around 37 C, and it is possible that some of the chlorambucil passes into the cocoa butter before the melted material is separated from the porous wafer surface, promoting distribution.

Graph 11b, of FIG. 1b, shows release of amitriptyline when the porous silicon is coated, and graph 12b shows release of amitriptyline when the porous silicon is uncoated. The rate of release is slower when the wafer has been coated, relative to the uncoated state. This effect may result from the repulsion, at a molecular level, between the hydrophilic amitriptyline and the hydrophobic cocoa butter. The cocoa butter may act as a barrier that tends to retain the amitriptyline in the pores, even when it has melted. Graph 11c, of FIG. 1c, shows release of neutral red when the porous silicon is coated, and graph 12c, shows release when the porous silicon is uncoated. The results are similar to those yielded by amitriptyline, and may be rationalised in the same way, since neutral red is also a hydrophilic substance.

The decrease of the initial release rate, shown in FIGS. 1b and 1c, may offer a particular advantage. If a hydrophilic drug were to pass into a patient's system too rapidly, this may have undesirable effects. In general, such a rapid release is more likely to happen for a hydrophilic drug, than for a hydrophobic drug.

The uncoated samples, the results for which are shown in FIG. 1, were prepared, with the steps (a) to (d) of method A.

Graph 21a, of FIG. 2a, shows the accumulative release for porous silicon that has been solution loaded with chlorambucil and cocoa butter. The rate of release for porous silicon that has been loaded with chlorambucil alone, shown in graph 22a, is faster than when the excipient is present. Similar results are shown in FIG. 2b for amitriptyline hydrochloride. Graph 21b corresponds to porous silicon solution loaded with amitriptyline and cocoa butter, and 22b corresponds to loading with amitrriptyline alone. These results may be explained by the hydrophobic cocoa butter being retained, to a large extent, in the pores of the porous silicon, even when it has melted, during the duration of the FIG. 2 experiment. Such retention may occur as a result of attraction to the porous silicon which has a hydrophobic surface. While the excipient remains in the pores, it appears to act as an, at least partial, barrier to release of the beneficial substance.

Graph 31, of FIG. 3, shows the accumulative release of chlorambucil from a pellet comprising an equal mass of cocoa butter and porous silicon. Graph 32, of FIG. 3, shows the accumulative release of chlorambucil from a pellet comprising no excipient. The rate of release of the chlorambucil is higher, from the excipient free sample, during the first seven hours. Beyond seven hours, the rate of release from the excipient containing pellet accelerates until it is higher than that of the excipient free sample. The pellets may comprise at least some bonded porous silicon particles, the spaces between which forming macropores that are occupied by the cocoa butter. During the initial stages of the experiment the cocoa butter may act as a barrier to the release of the chlorambucil. During the later stages, the pellet comprising the cocoa butter was observed to fragment, which may account for the acceleration of drug release. The hydrophobic nature of both the porous silicon employed and the cocoa butter, may result in retention of the excipient in the macropores between the bonded porous silicon particles, even after it has melted. Step (d) is omitted from Method C in order to prepare the excipient free sample.

1. A pharmaceutical product comprising porous silicon, a beneficial substance, and an excipient, the beneficial substance being located in at least some of the pores of the porous silicon, and the excipient having a structure and composition such that it has a melting point between 25 C and 45 C.

2. A pharmaceutical product according to claim 1 characterised in that the excipient and the porous silicon are arranged such that the excipient forms a barrier between at least some of the beneficial substance and the exterior of the pharmaceutical product.

3. A pharmaceutical product according to claim 1 characterised in that at least some of the exciptient is located in at least some of the pores of the porous silicon.

4. A pharmaceutical product according to claim 1 characterised in that the porous silicon comprises a multiplicity of covalently bonded porous silicon particles, each covalently bonded porous silicon particle being covalently bonded to at least one of the other covalently bonded porous silicon particles.

5. A pharmaceutical product according to claim 1 characterised in that the porous silicon is obtainable by anodisation and/or stain etching.

6. A method of delivering a beneficial substance to an animal or human comprising the steps:
(c) introducing a pharmaceutical product, according to claim 1, to a human or animal; and
(d) allowing the excipient to melt thereby delivering the beneficial substance.

7. A method of loading a hydrophilic beneficial substance into at least some of the pores of porous silicon, at least part of the surface of the porous silicon having a composition and structure such that it is hydrophobic, the method comprising the steps: (a) combining the hydrophilic beneficial substance with a hydrophobic excipient to produce an excipient mixture, (b) melting the excipient mixture, (c) allowing the molten excipient mixture to pass into the pores of the porous silicon.
